(19) United States
(12) Patent Application Publication
(10) Pub. No.: US 2016/0144069 A1
(43) Pub. Date: May 26, 2016

(54) SUTURABLE HYBRID SUPERPOROUS HYDROGEL KERATOPROSTHESIS FOR CORNEA

(71) Applicant: The Board of Trustees of the University of Illinois, Urbana, IL (US)

(72) Inventors: Michael Cho, Naperville, IL (US); Amelia Zellander, Chicago, IL (US)

(21) Appl. No.: 15/012,110

(22) Filed: Feb. 1, 2016

Related U.S. Application Data
(63) Continuation-in-part of application No. 13/284,301, filed on Oct. 28, 2011.
Application No. 13/284,301 is a continuation-in-part of application No. 12/511,145, filed on Jul. 29, 2009, now abandoned.
(60) Provisional application No. 61/085,064, filed on Jul. 31, 2008.

(51) Int. Cl. A61L 27/26; A61L 27/52; A61L 27/56

(52) U.S. Cl. CPC A61L 27/26; A61L 27/56; A61L 27/52; A61L 2430/16; A61L 2400/18

(57) ABSTRACT

The present invention features a superporous hydrogel scaffold for corneal regeneration or replacement and a method for producing the same. The superporous hydrogel is composed of a poly(2-hydroxyethyl methacrylate) (PHEMA) and poly(methyl methacrylate) (PMMA) copolymer mixed with collagen. The scaffold can be used as a suturable hybrid corneal implant or keratoprosthesis.

### INTRODUCTION

This application is a continuation-in-part of U.S. Ser. No. 13/284,301, filed Oct. 28, 2011, which is a continuation-in-part of U.S. Ser. No. 12/511,145 filed Jul. 29, 2009, now abandoned, which claims the benefit of U.S. Provisional Application No. 61/085,064, filed Jul. 31, 2008, which are herein incorporated by reference in their entirety.

### BACKGROUND OF THE INVENTION

The cornea is an avascular and optically transparent tissue that refracts and filters light rays before they enter the eye. A clear cornea is essential for clear vision. The cornea may become opacified following injuries, degenerations, or infections. The Vision Share Consortium estimates that corneal blindness affects more than 10 million patients worldwide (Carlsson, et al. (2003) Curr. Opin. Ophthalmol. 14(4):192-7). The gold standard treatment is surgical replacement of the cornea using freshly donated cadaver human corneas. Currently, about 40,000 corneal transplants are performed each year in the United States (Eye Bank Association of America. Statistical report 2000), with a 2-year success rate as high as 90% for uncomplicated first grafts performed in nonvascularized “low-risk” patients (Council on Scientific Affairs (1988) JAMA 259:719; The Collaborative Corneal Transplantation Research Group. (1992) Arch. Ophthalmol. 110:1392). However, the success in low-risk corneal transplantation contrasts sharply with the results of corneal grafts placed in so-called “high-risk” patients in which rejection rates can increase up to 50-70%, even with maximal local and systemic immune suppression (Mader & Stulting (1991) Ophthalmol. Clin. North Am. 4:411; Foulks & Sanfilippo (1982) Am. J. Ophthalmol. 94(5):622-9). Immune-rejection still remains the leading cause of corneal transplant failure (Ing, et al. (1998) Ophthalmology 105(10):1855-65). The risk factors for immunologic rejection include previous graft rejection, corneal vascularization, and young age. These “high risk” patients typically undergo repeated surgeries resulting it excessive pain, cost, and use of limited resources. For instance, it is not uncommon for an infant born with corneal disease to undergo 15-20 corneal transplants by the time they reach adulthood with each graft lasting only 3-6 months before being succumbed to rejection. Therefore, there is a distinct need for alternative treatments that circumvent rejection in these high risk patients (Coster & Williams (2003) Eye 17(8):996-1002).

Artificial cornea or keratoprosthesis were designed to meet the unmet market need for corneal replacement. A notable amount of clinical data is available for two artificial corneas, the Boston Keratoprosthesis (KPro) and AlphaCor. The Boston KPro Type I is a partially artificial KPro. It includes a skirt made of human corneal tissue and a core made of PMMA. The type II version of Boston KPro is fully artificial, made of PMMA. Boston KPros are widely used clinically. AlphaCor is a fully artificial PHEMA based core-skirt KPro. Retention for Boston KPro Type I is 95% after 8.5 months (Zerbe, et al. (2006) Ophthalmology 113(10):1779.e1-1779.e7). For Alphaccor, retention rates are reportedly 92% after 6 months (Hicks, et al. (2006) Cornea 25(9):1034-42). However, a lack of stable host integration was observed with Alphacor (Chirila (2001) Biomaterials 22(24):3311-7). Other designs also inadequately address one or more of the vital parameters for an ideal keratoprosthesis, i.e., host integration, mass transport, tissue epithelialization or innervations (Guo, et al. (2007) Zhonghua Yan Ke Za Zhi (2007) 43:602-607; Kim, et al. (2007) Cornea 26:546-55; Gomaa, et al. (2010) Clin. Exp. Ophthalmol. 38:211-224; June, et al. (2012) Chapter 10: Keratoprosthesis. In: Krieglstein & Weinreb, ed. Cornea and External Eye Disease: Essentials in Ophthalmology. p. 137-144; Griffith, et al. (2005) Essentials in Ophthalmology: Cornea and External Eye Disease. Springer, p. 37-53. Inadequate keratoprosthesis design can result in extrusion, tissue necrosis, increased intraocular pressure or infection.

Many methods have been employed to engineer 3-D porous scaffolds including salt leaching, freeze drying, and layer by layer lithography using heat, adhesives, light, or molds. While these methods have advantages, drawbacks include difficulty in achieving interconnected pores, toxic byproducts, difficulty incorporating cells, or long processing times (Tsang & Bhatia (2004) Adv. Drug Deliv. Rev. 56(11):1635-4).

U.S. Pat. No. 6,960,617 describes the use of hydrogels with improved elasticity and mechanical strength. The hydrogels taught are superporous and are used to form a network of polymer chains. The patent does not teach or suggest combining any other compound with the hydrogel in order to improve the function or biocompatibility of the polymer.

While the pores provide a physical pathway for cellular migration from host to implant, they do not provide biological cues for cells to adhere, survive and secrete extracellular matrix. A cell adhesion component is required. For example, collagen matrices support cell growth and differentiation (Sun, et al. (2004) Tissue Eng. 10(9-10):1548-57; Yoneno, et al. (2005) J. Biomed. Mater. Res. A 75(3):733-41; Reyes & Garcia (2004) J. Biomed. Mater. Res. A 69(4):591-600). Many different types of biosynthetic matrices are described by others. For example, U.S. Publication No. 2004/0048796 teaches the use of collagen biofabric for medical and surgical applications. The collagen biofabric is prepared from a placental membrane preferably human, by decellularizing the amniotic membrane. U.S. Publication No. 2006/0083773 discloses artificial corneal implants designed to replace or augment the cornea. The implants disclosed are fabricated from a double network hydrogel that consists of biocompatible polymers, wherein at least one of the network polymers is based on a hydrophilic polymer, wherein the implant has epithelialization promoting biomolecules that are covalently linked to the surface of the double network hydrogel. The implant also a physiologic diffusion coefficient to allow passage of nutrients to the adhered cells. U.S. Publication No. 2006/0246113 teaches use of a biosynthetic matrix comprising a hydrogel that is formed by chemically cross-linking a synthetic polymer and a biopolymer. EP 1 741 457 discloses a biosynthetic matrix comprising a hydrogel which is formed by cross-linking a synthetic polymer and a biopolymer as well. However, none of the matrices described in the prior art has been successfully used to produce a corneal implant material with sufficient strength and biocompatibility for use in corneal replacement surgery. There remains a need for materials that can be used in corneal replacement surgery.

### SUMMARY OF THE INVENTION

The present invention is a scaffold for corneal regeneration or replacement including a superporous hydrogel matrix composed of a poly(2-hydroxyethyl methacrylate) (PHEMA) and poly(methyl methacrylate) (PMMA) copolymer, and a cell adhesion agent such as collagen included in the pores of the superporous hydrogel matrix. In certain embodiments, the pores of the superporous hydrogel matrix have an average diameter of greater than 100 μm and are connected to one another to form an open channel system. In other embodiments, the equilibrium swelling capacity of the superporous hydrogel matrix ranges from 50 to 500 g/g and the superporous hydrogel matrix has a swelling rate of less than one minute. In particular embodiments, the superporous hydrogel matrix has an ultimate tensile strength of about 80 kPa or more. In another embodiment, the copolymer is formed by dissolving HEMA and MMA monomers in a solvent and polymerizing the monomers at a temperature below 45° C. in the presence of a pore-forming agent. A suturable implant including the scaffold and a method for producing the scaffold are also provided.

### BRIEF DESCRIPTION OF THE DRAWINGS

FIG. 1 depicts the results of tensile strength testing on polymers of the instant invention. Porous PHEMA-PMMA copolymer, porous PHEMA-PEGDA copolymer and porous PEGDA polymer were compared in the testing. All materials were tested in their hydrated state. PHEMA-PMMA copolymer showed significantly greater tensile strength as compared to the PEGDA polymer as well as a copolymer of PHEMA-PEGDA.

### DETAILED DESCRIPTION OF THE INVENTION

The present invention is a hybrid scaffold composed of a cell adhesion agent combined with a PHEMA-PMMA (poly(2-hydroxyethyl methacrylate)-poly(methyl methacrylate)) superporous hydrogel (SPH). This invention provides a method for complete 3-D cell adhesion and encourages cell ingrowth, while maintaining the overall mechanical strength of the SPH. Using this scaffold, the present invention is also a suturable hybrid implant. The suturable hybrid implant comprises a PHEMA-PMMA copolymer and cell adhesion agent. The hybrid implant provides for promotion of host integration and mass transport in vivo and can be used as the skirt in a core-skirt keratoprosthesis for corneal implant. The skirt-core keratoprosthesis model for corneal implants is one where the core permits vision while the skirt facilitates stable host integration. Also provided by the present invention is a method for producing a suturable hybrid implant which comprises mixing in an aqueous solution methylmethacrylate (MMA), 2-hydroxyethyl methacrylate (HEMA), deionized water, pentaerythritol tetraacrylate, and diemthylformamide to form a superporous PHEMA-PMMA hydrogel solution; cooling the superporous PHEMA-PMMA hydrogel solution; adding collagen to the cooled superporous PHEMA-PMMA hydrogel solution to form a collagen-hydrogel solution; and incubating the collagen-hydrogel solution at 37° C. to create a suturable hybrid implant.

Outcomes for synthetic KPro skirts indicate the need for alternative approaches that enable longer term use and maintain a full visual field. The Seoul Type Keratoprosthesis fails to provide both sufficient host integration and full visual field. It is composed of a PMMA optic and a skirt made of either polyurethane or polypropylene. Use of this device has resulted in a 66.7% anatomic retention rate at 68 months. Such devices develop corneal melt leading to full exposure of the skirt. In another example, the Stanford KPro, composed of a hybrid network of poly(ethylene glycol) and poly(acrylic acid) (PEG/PAA) in its central optic component, was tolerated well in an animal model for up to 6 weeks (Griffith, et al. (2005) In Essentials in Ophthalmology: Cornea and External Eye Disease, Chapter 3. T. Rheinhard (ed). Springer). However, evidence of host integration was not presented. Alphacor, previously used in clinical studies, resulted in unfavorable outcomes for some patients. (Griffith, et al. (2005) In Essentials in Ophthalmology: Cornea and External Eye Disease, Chapter 3. T. Rheinhard (ed). Springer; June, et al. (2010) In Cornea and External Eye Disease: Essentials in Ophthalmology, Chapter 10. Weinreb & Krieglstein (eds.), pp. 137-144). Alphacor retention at the 2 year follow up has been reported to be up to 62%, and topical use of medroxyprogesterone (MPG) postoperatively was found to be associated with fewer corneal stromal melts, the most frequent complication. (Gomaa, (2010) Clin. Exp. Ophthalmol. 38:211-224). Yet, other Alphacor complications have included retroprosthetic membrane formation, optic damage, and poor biointegration. (Sheardown (2008) In Regenerative Medicine in the Cornea, pp. 1060-1071). Moreover, wound dehiscence due to Alphacor's inability to hold sutures was a common mode of failure during preclinical trials (Hicks (1997) Austral. NZ J. Ophthalmol. 25:S50-S52) Finally, a tensile modulus in the 3-14 KPa range has been observed for PHEMA sponges created using various crosslinkers, including ethylene dimethacrylate (EDMA) which is used in the preferred formation of a keratoprosthesis design patented by Chirila et. al (U.S. Pat. No. 5,458,819; Lou (2000) J. Mater. Sci. 11:319-325).

The present invention was developed to address drawbacks to existing synthetic KPros. The present hybrid construct (porous PHEMA-PMMA with cell adhesion agent) is designed to hold sutures and promote cell migration which leads to tissue development and the integration of the skirt into the host's ocular tissue.

The present invention was developed by first investigating the use of a hybrid scaffold composed of collagen intertwined in a polyethylene glycol diacrylate (PEGDA)-based superporous hydrogel (SPH). The present invention has now been extended to use other SPH polymers; it encourages cell ingrowth while maintaining the overall mechanical strength of the SPH. The hybrid scaffold is produced by dehydrating the SPH, then reswelling in a collagen-cell solution to create a hybrid scaffold. The scaffold produced by the method of the present invention is one with strong bulk properties, yet natural 3-D cell adhesive properties. Indeed, while collagen increases cell adhesion, retention, and ingrowth, the overall mechanics of the hybrid are not dependent on collagen and greatly resembles the superporous hydrogel. Thus, the hybrid superporous hydrogel provides mechanical stability and interconnected pores while the collagen provides adhesive binding sites.

Experiments were performed with the PEGDA-based hybrid to demonstrate that the hybrid scaffolds of the present invention could be successfully used in vivo. In these experiments, two rats were implanted with artificial corneas comprising either the hybrid scaffold (SPH and collagen) or a scaffold of hydrogel without collagen (no hybrid). The novel nature of the artificial corneas produced using the hybrid scaffold was related to the hybrid skirt (outer) portion of the implant. Thus, while the central portion of the implant maintained clarity, the peripheral skirt was designed to encourage integration with the tissue of the eye. To test the degree of in vivo integration of this type of corneal implant, a peripheral skirt was implanted into rat corneas and observed after 2 weeks. Examination of the implants showed that there were differences depending on the nature of the implant. The implant with a peripheral skirt containing the hybrid material (SPH and collagen) was less noticeable in the eye after 2 weeks. The hybrid skirt implant was well tolerated and biocompatible. Further, the results indicated that the hybrid skirt implant was better integrated with the surrounding eye tissue as compared to the implant without collagen.

In an alternative formulation, the present invention is a superporous hydrogel comprising a PHEMA-PMMA copolymer. This copolymer has now been shown to have improved properties when incorporated into a keratoprosthesis as compared to a PEGDA-based superporous hydrogel. The present invention also provides for a novel method for forming a superporous hydrogel for application as a keratoprosthesis. In the method of the present invention, the superporous hydrogel is formulated as a PHEMA-PMMA hybrid copolymer. The method of the present invention involves mixing methyl methacrylate (MMA), 2-hydroxyethyl methacrylate (HEMA), pentaerythritol tetraacrylate (PETA), ammonium persulfate, N,N,N′,N′-tetramethylethylenediamine (TEMED), solvent (e.g., DMF), and deionized water to form a gel solution. The use of PETA in the gel solution provides for crosslinking of the PHEMA-PMMA copolymers to produce a gel with the desired strength and mechanical properties and is a novel feature of the method of forming the superporous hydrogel of the instant invention. The use of an organic solvent such as DMF to dissolve PHEMA-PMMA in the gel solution is another novel feature of the present method of forming the superporous hydrogel of the present invention as it provides for dissolution of the PHEMA and PMMA while not destroying large pores that are an important feature of the PHEMA-PMMA copolymer. Stirring during the polymerization is a key step that leads to formation of large pores. The pores can be made larger by stirring the mixture up to a threshold temperature. Once the threshold temperature has been reached, the gel solution that results from the method of the present invention develops a viscosity that is flexible enough to be placed into a contact lens-shaped mold, mimicking the curvature of the cornea. Once the gel has been produced, the formation of an inter-penetrating collagen network in the porous PHEMA-PMMA copolymer can take up to 36 hours due to the slow expansion of PHEMA-PMMA in aqueous solution. Once the PHEMA-PMMA copolymer has been fully expanded in a collagen solution, the construct that results is a suturable hybrid keratoprosthesis for implantation. The construct can be used as is or subjected to post-processing (e.g., washing) to remove excess collagen. The suturability of the copolymer of the instant invention is an important advance over other materials. Experiments have been performed showing that the PHEMA-PMMA copolymer is mechanically stable in solution such as deionized water and phosphate buffered saline (PBS), while the copolymer is elastic enough to allow for pulling on the material with moderate force. Moreover, the collagen network of the hybrid suturable keratoprosthesis of the present invention facilitates migration of cells into the construct from the host. When examined through staining with monoclonal anti-collagen type I antibody, collagen fibers were shown to have formed within the pores of the PHEMA-PMMA implant. When stained to identify live cells (calcein-AM staining) versus dead cells (ethidium homodimer-1), cell migration into the suturable hybrid keratoprosthesis was clearly evident.

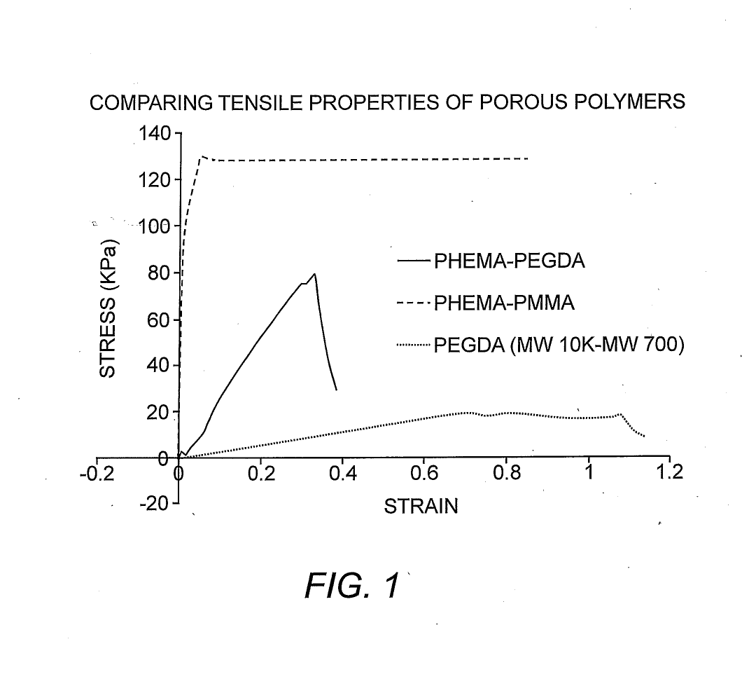

Experiments were performed to examine the tensile strength of the PHEMA-PMMA copolymer as compared to other polymers. All materials were tested in their hydrated state. As shown in FIG. 1, the tensile strength of the PHEMA-PMMA copplymer of the present invention has a tensile strength that is one to two orders of magnitude greater than the PEGDA polymer. As would be expected based on these findings regarding material strength, PEGDA polymer (MW 10K-MW 700) is a soft material that can be made porous to accommodate cell ingrowth but cannot be successfully sutured. PHEMA-PEGDA copolymer is more mechanically stable than PEGDA, but also lacked the strength for suturability. In contrast, PHEMA-PMMA copolymer maintains its integrity under moderate to mildly forceful tension, and also resists rupture following the insertion of a needle and the subsequent application of force. Thus, the PHEMA-PMMA copolymer of the present invention provides for a suturable keratoprosthesis that is also capable of stable host integration.

Thus, the porous PHEMA-PMMA copolymer with a collagen type 1 network embedded in the construct of the present invention represents a vastly improved artificial cornea (keratoprosthesis). The present invention addresses the two major criteria that are not met by existing artificial corneas: cell migration/host integration and structural/mechanical stability that allows for suturing of the prosthesis. Data shows that the hybrid keratoprosthesis of the present invention promotes cell ingrowth, and also exhibits the tensile strength needed for suturing and implantation. As a result, the present invention provides a novel keratoprosthesis that could support full or partial thickness cornea replacement surgeries. Accordingly, the present invention provides hybrid scaffolds composed of collagen and cells incorporated into the pores of a superporous hydrogel and a method for producing the same.

A superporous hydrogel, as known in the art and used herein, refers to a semi-solid three-dimensional structure which is capable of absorbing a substantial amount of water. Superporous hydrogels are distinguished from other porous hydrogels (e.g., macroporous and microporous hydrogels) by the nature of the pores and methods to generate the pores (e.g., polymers and polymerization conditions). In particular, whereas hydrogels with effective pore sizes in the 10-100 nm range and in the 0.1-10 μm range are defined as microporous and macroporous hydrogels, respectively, the pore sizes in superporous hydrogels are larger than 100 μm, usually in the range of several hundred micrometers. See Polymeric Drugs & Drug Delivery Systems (2001) Ottenbrite & Kim, ed. CRC Press, Boca Raton, Fla., pg. 147-150; and EP 0988108. In particular embodiments, the pores of the superporous hydrogel of this invention have an average diameter of greater than 100 μm. In other embodiments, the pores of the superporous hydrogel of this invention have an average diameter less than or equal to about 600 μm or 300 μm. In certain embodiments, the pores of the superporous hydrogel of this invention have an average diameter in the range of 100-300 μm.

In addition to pore size, pore connectivity distinguishes superporous hydrogels from other porous hydrogels. In particular, whereas microporous and macroporous hydrogels are referred to as polymer “sponges” based upon their water absorbing characteristics, pores of superporous hydrogels are connected to one another to form an open channel system (i.e., the pores are interconnected) such that swelling of superporous hydrogels is by capillary wetting rather than by diffusion. In this respect, the equilibrium swelling capacity in superporous hydrogels ranges from 50 to 500 g/g and can be reached in less than one minute, or more preferably in less than 30 seconds. In particular embodiments, the swelling rate is between 5 and seconds. Superporous hydrogels can be produced as disclosed herein, i.e., a foaming reaction optimized for simultaneous polymerization, or any other suitable method employed in the art for producing superporous hydrogels including, but not limited to salt-phase separation (Chirila, et al. (1993) Biomaterials 14:26-38; Barvic, et al. (1967) J. Biomed. Mater. Res. 1:313-323; Dusek, et al. (1969) Coll. Czech. Chem. Commun. 34:136-157), and the use of porosigens (Oxley, et al. (1993) Biomaterials 14:26-38; Krauch, et al. (1968) Natur. Wissenscheften 55:539-540). As is appreciated by the skilled artisan, the size of the pores can be regulated by, e.g., the nature of the solvent or solvents in which the gel is formed and/or the amount of polymerization initiator or catalyst. In particular embodiments, the entire polymerization reaction takes place at temperatures below 45° C., preferably between 25 and 40° C., or more preferably at about 37° C.

In so far as uptake of material is based on capillary action rather than diffusion (Gemeinhart, et al. (2000) J. Biomater. Sci. Polym. Ed. 11(12):1371-80; Gemeinhart, et al. (2001) J. Biomed. Mater. Res. 55(1):54-62), the instant superporous hydrogels can rapidly incorporate a variety of soluble materials, such as cells and proteins, within the pores of the scaffold matrix. Superporous hydrogels are composed of polymers that will swell, without dissolving, when placed in water or other biological fluids. Hydrogels can generally absorb a great deal of fluid and, at equilibrium, typically are composed of 60-90% fluid and only 10-30% polymer. Hydrogels are particularly useful due to the inherent biocompatibility of the cross-linked polymeric network (Hill-West, et al. (1994) Proc. Natl. Acad. Sci. USA 91:5967-5971). Hydrogel biocompatibility can be attributed to hydrophilicity and ability to imbibe large amounts of biological fluids (Brannon-Peppas. Preparation and Characterization of Cross-linked Hydrophilic Networks in Absorbent Polymer Technology, Brannon-Peppas and Harland, Eds. 1990, Elsevier: Amsterdam, pp 45-66; Peppas and Mikos. Preparation Methods and Structure of Hydrogels in Hydrogels in Medicine and Pharmacy, Peppas, Ed. 1986, CRC Press: Boca Raton, Fla., pp 1-27). Also, hydrogels closely resemble the natural living extracellular matrix (Ratner and Hoffman. Synthetic Hydrogels for Biomedical Applications in Hydrogels for Medical and Related Applications, Andrade, Ed. 1976, American Chemical Society: Washington, D.C., pp 1-36).

Hydrogel matrices of the invention are composed of synthetic hydrophilic polymers which have been synthetically produced and which are hydrophilic, but not necessarily water-soluble. Examples of synthetic hydrophilic polymers which can be used in the practice of the present invention are polyethylene glycol (PEG); polyoxyethylene; polymethylene glycol; polytrimethylene glycols; polyvinylpyrrolidones; poly(acrylic acid); poly(itaconic acid); poly(methacrylic acid); poly(hydroxypropyl acrylamide) (HPMA); poly(peptides) such as polyglutamate, polylysine, polyaspartate, polyserine, polythreonine, polycysteine; and polyoxyethylene-polyoxypropylene block polymers; and copolymers, and derivatives and mixtures thereof. While natural marine biopolymers such as agarose, chitosan, and alginate are also embraced by the present invention, in some embodiments, the hydrophilic matrix is not a naturally occurring polymer such as a protein, starch, cellulose, heparin, or hyaluronic acid. In a preferred embodiment, the hydrogel matrix is a poly(methacrylic acid) polymer or similar hydrophilic polymers such as poly(hydroxyethoxyethyl methacrylate)(PHEEMA), poly(hydroxydiethoxyethyl methacrylate)(PHDEEMA), poly(methoxyethyl methacrylate)(PMEMA), poly(methoxyethoxyethyl methacrylate)(PMEEMA), poly(methoxydiethoxyethyl methacrylate)(PMDEEMA) and poly(ethylene glycol dimethacrylate)(PEGDMA). In a more preferred embodiment, the poly(methyacrylic acid) polymer is a PHEMA-PMMA copolymer.

Although different synthetic hydrophilic polymers and selected biopolymers can be used in connection with forming the hydrophilic matrix of the invention, the polymer must be biocompatible and hydrophilic, but crosslinked physically or chemically to prevent dissolution. Particularly suitable polymers include those which are extensively used in the modification of biologically active molecules because they lack toxicity, antigenicity, and immunogenicity; have a wide range of solubilities; are generally non-biodegradable and are easily excreted from most living organisms including humans.

Poly(ethylene glycol) diacrylate (PEGDA) and poly(methacrylic acid) hydrogels have been widely accepted in many biomedical applications (Peppas, et al. (1999) J. Controlled Release 62:81-87. Such hydrogels are hydrophilic, biocompatible, nontoxic, and exhibit variable mesh size depending upon macromer length. As exemplified herein, superporous hydrogels produced from PEGDA and poly(methacrylic acid) are not toxic to cells and can be readily produced using the gas foaming method. Moreover, the hydrogels of the present invention are optically clear rendering them ideal clarity in vivo. Accordingly, particular embodiments of the present invention embrace superporous hydrogels produced with PEGDA or poly(methacrylic acid) polymers, such as PHEMA and PMMA.

Superporous hydrogels of the present invention can be further modified to possess high mechanical strength by incorporating a cross-linked hydrophilic agent such as sodium alginate, pectin, chitosan, or (polyvinyl) alcohol that can crosslink after the matrix is formed (Omidian, et al. (2006) Macromol. Biosci. 6:703-10). Hydrogels can also be made degradable in vivo by incorporating PLA, PLGA or PGA polymers. Moreover, superporous hydrogels can be modified with fibronectin, laminin, vitronectin, or, for example, RGD for surface modification, which can promote cell adhesion and proliferation (Heungsoo Shin (2003) Biomaterials 24:4353-4364; Hwang, et al. (2006) Tissue Eng. 12:2695-706). Indeed, altering molecular weights, block structures, degradable linkages, and cross-linking modes can influence strength, elasticity, and degradation properties of the instant hydrogels (Nguyen & West (2002) Biomaterials 23(22):4307-14; Ifkovits & Burkick (2007) Tissue Eng. 13(10):2369-85).

In particular embodiments, the superporous hydrogel matrix of the invention has an ultimate tensile strength of about 80 kPa or more. As used herein, the term “ultimate tensile strength” refers to the maximum resistance to fracture of a material (e.g., the superporous hydrogel matrix) under tensile stress. The ultimate tensile strength can be measured in accordance with the procedures outlined in ASTM Standard D638-03 (Type V) and is considered to be within the ranges set forth herein when so determined by any of the procedures in ASTM Standard D638-03 (Type V). Desirably, the superporous hydrogel matrix of the invention has an ultimate tensile strength of about 90 kPa or more. More particularly, the superporous hydrogel matrix of the invention has an ultimate tensile strength of about 100 kPa or more, or most preferably in the range of 100 to 140 kPa.

Superporous hydrogels can also be modified with functional groups for covalently attaching a variety of proteins (e.g., collagen) or compounds such as therapeutic agents. Therapeutic agents which can be linked to the matrix include, but are not limited to, analgesics, anesthetics, antifungals, antibiotics, anti-inflammatories, anthelmintics, antidotes, antiemetics, antihistamines, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary anti-infectives, vasoconstrictors, vasodilators, vitamins, xanthine derivatives, and the like. The therapeutic agent can also be other small organic molecules, naturally isolated entities or their analogs, organometallic agents, chelated metals or metal salts, peptide-based drugs, or peptidic or non-peptidic receptor targeting or binding agents. It is contemplated that linkage of the therapeutic agent to the matrix can be via a protease sensitive linker or other biodegradable linkage.

In addition to functional groups, the polymers of the instant hydrogels can further contain a means for controlled biodegradation to facilitate removal of the matrix polymer from the subject being treated. For example, hydrogels can be made to biodegrade at a faster rate by modification (Sawhney, et al. (1994) J. Biomed. Mater. Res. 28:831-838). Hydrogels can be made biodegradable by incorporating a biodegradable cross linker or by utilizing biodegradable copolymers (Sawhney, et al. (1993) Macromolecules 26:581-587; Park, et al. Biodegradable Hydrogels for Drug Delivery. 1993, Lancaster, Pa.: Technomic Pub. ix, 252; Watanabe, et al. (2002) Biomaterials 23:4041-4048; Yamini, et al. (1997) J. Macromol. Sci. A34:2461-2470). For example, telechelic biodegradable block copolymers, specifically degraded by either plasmin or crude collagenases, have been used in cross-linked hydrogels (West, et al. (1999) Macromolecules, 32:241-244). The extent and rate or degradation is controlled by the specific degradation mechanism used thereby limiting accumulation of the matrix at the site of implantation.

As indicated, hydrogels of the invention can be produced by conventional gas foaming methods or phase separation methods, wherein a hydrogel precursor solution is prepared and a pore-forming agent is added thereto such that the monomers of the precursor solution gel or polymerize to form a matrix with a plurality of interconnected macropores dispersed therein. A precursor solution is defined as the mixture of components which are combined to produce the superporous hydrogel structure, but lacks a pore-forming agent. A precursor solution of the invention can include, but is not limited to, one or more hydrophilic monomers, an initiator, and a foam stabilizer. Suitable hydrophilic polymers are disclosed herein. Suitable initiators include, e.g., APS/TEMED and a suitable foam stabilizer can be a block copolymer such as PLURONIC F-127. A pore-forming agent can be a chemical or physical foaming agent. In some embodiments, the pore-forming agent is a chemical foaming agent such as sodium bicarbonate. In other embodiments, the pore-forming agent is a physical foaming agent gas, e.g., compressed air or nitrogen. In further embodiments, the pore-forming agent is a salt used in a phase separation method known to those of skill in the art.

In accordance with the method for producing a superporous hydrogel of the invention, the superporous hydrogel matrix is dehydrated. The hydrogel matrix can be dehydrated by any suitable chemical and/or physical means. For example, dehydration can be achieved using a combination of alcohol (e.g., ethanol) and a dehydrator.

In a preferred embodiment, the present invention is a method for producing a superporous PHEMA-PMMA hydrogel which comprises mixing in a solution methylmethacrylate (MMA), 2-hydroxyethyl methacrylate (HEMA), deionized water, pentaerythritol tetraacrylate (PETA), and a solvent such as DMF to form a superporous PHEMA-PMMA hydrogel, wherein the solvent promotes dissolution of MMA and HEMA into a gel solution and PETA promotes crosslinking of the PHEMA-PMMA copolymer. In certain embodiments, the solvent is DMF. In other embodiments, the solvent is dimethyl sulfoxide (DMSO), propanol, glycerol, ethylene glycol, cyclohexanol, toluene or a mixture thereof. In certain embodiments, the solvent is less than 10% by weight of the polymerization reaction. In a more preferred embodiment the method of the present invention involves mixing 10% v/v MMA, 45% v/v HEMA, 5 mg PETA, 2 mg ammonium persulfate, 10 μl N,N, N′,N′-tetramethylethylenediamine (TEMED), 6% v/v DMF, and 22% deionized water.

To incorporate a molecule of interest (e.g. a cell adhesion agent such as collagen), with or without cells embedded therein, the hydrogel matrix is reswelled or rehydrated in a solution containing the molecule of interest with or without the cells. Molecules which can be incorporated into the pores of the superporous hydrogel matrix include, but are not limited to, vitamins and other nutritional supplements; cell adhesion agents including proteins such as collagen, fibronectin and laminin; peptides and proteins; carbohydrates (both simple and/or complex); proteoglycans; antigens; oligonucleotides (sense and/or antisense DNA and/or RNA); antibodies (for example, to infectious agents, tumors, drugs or hormones); and gene therapy reagents. In some embodiments, the molecule of interest is cell adhesion agent. In other embodiments, the cell adhesion agent is collagen. In particular embodiments, the collagen is Type I collagen. Desirably the molecule of interest is in a biologically compatible solution, i.e., a solution which is non-toxic in vivo. Suitable solutions include, but are not are limited to, water, saline, a buffer and the like.

Type I collagen is the most abundant collagen of the human body. It is present in scar tissue, tendons, and the organic part of bone. Type II collagen is a component of articular cartilage and is found in association with Type IX collagen, whereas Type III collagen is the collagen of granulation tissue, and is produced quickly by young fibroblasts before the tougher type I collagen is synthesized. Type XII collagen is found to interact with types I and III collagen. Type IV collagen is part of the basal lamina. Type V and Type VI collagen are components of most interstitial tissue and are associated with type I collagen. Type VII collagen is a component of the epithelia as is Type VIII collagen. Type X collagen is hypertrophic and part of mineralizing cartilage, whereas Type XI collagen is a component of cartilage. Therefore, depending on the site of implantation and the intended therapeutic result, one or more collagens can be incorporated into the pores of the superporous hydrogel matrix. Collagens can be obtained in solution as a pepsin-solubilized collagen dissolved in acid (e.g., Vitrogen; ANGIOTECH® Biomaterials, Palo Alto, Calif.). The collagen can be neutralized (e.g., to pH 7.0 to pH 7.4 with NaOH), and directly incorporated into the superporous hydrogel matrix or combined with a cell of interest and be incorporated into the superporous hydrogel matrix. The collagen can than be solidified via fibrillogenesis (e.g., at 24° C. to 37° C. in the presence or absence of CO₂) with cells suspended therein. In a preferred embodiment, type I collagen is incorporated into the pores of the superporous PHEMA-PMMA hydrogel of the present invention. This is accomplished by adding collagen to a cooled PHEMA-PMMA hydrogel solution (approximately 2-8° C.), wherein the solution remains cooled throughout the collagen absorption process. Once the collagen has incorporated into the PHEMA-PMMA solution, the collagen solution is incubated at 37° C. for one hour in a cell incubator. The resulting product is a suturable hybrid implant or a suturable hybrid keratoprosthesis.

When it is desired that cells be incorporated into the instant superporous hydrogels, the cells can be combined with the solution containing the molecule of interest prior or after the solution has be used to reswell or rehydrate the hydrogel. In particular embodiments, the cells are added to the solution prior to reswelling the hydrogel. Types of cells of particular use in this invention include, but are not limited to, stem cells, fibroblasts, epithelial cells, endothelial cells, mesenchymal cells, insulin-producing islet cells, hepatocytes, myocytes, neurons, chondrocytes, skin cells, bone marrow cells, and the like. The cells can be autogenic, allogenic or xenogenic with respect to the subject receiving the instant hybrid superporous hydrogel. Cells can be isolated from biopsy samples or generated by differentiation and expansion of stems cells using conventional methods. In addition to being incorporated into the pores of the hydrogel matrix, some embodiments embrace encapsulation of cells within the hydrogel itself, e.g., by adding cells to the hydrogel precursor solution prior to polymerization. Cells encapsulated within the hydrogel matrix and hydrogel pores can be the same or different. For example, one could encapsulate stem cells in the hydrogel matrix and encapsulate cells capable of producing growth or differentiation factors in the pores, or vice versa.

In addition to cells incorporated into the pores of the hybrid superporous hydrogel, the present invention further embraces coating one or more surfaces of the hybrid superporous hydrogel matrix with one or more of the cell types disclosed herein. In particular, the present invention embraces attaching epithelial cells to the surface of the hybrid hydrogel matrix via a layer of collagen. In addition, the invention embraces the inclusion of a central core in the hybrid hydrogel which is filled with one or more optically clear macromers. For the purposes of the present invention, a macromer is optically clear when it can transmit light at wavelengths ranging from 200 nm to 1000 nm and has a reflective index of more than 1 or more desirably more than 1.3. Suitable macromers include, e.g., the hydrophilic polymers disclosed herein. In particular embodiments, the optically clear macromer is PEGDA.

As demonstrated herein, the components and fabrication method of the invention are not toxic to cells and enables the incorporation of cells within the pores of the superporous hydrogel. Such hydrogels find application as biological scaffolds for maintaining and growing cells and in the functional replacement of injured or damaged organs of the body. In certain embodiments, the instant hybrid superporous hydrogel is used in the preparation of a variety of formed implants for use in medical applications. Advantageously, the superporous hydrogel is designed to provide cells to a damaged or injured site to facilitate regeneration. Accordingly, the instant composition is useful for providing localized delivery of cells to a subject. Such delivery can be used to, e.g., promote wound healing and in tissue regeneration or replacement. In particular embodiments, the hydrogels of the present invention are used in tissue engineering or regenerative medicine, as a model organ system for drug testing, or for use in cell expansion.

In particular embodiments of this invention, the instant hybrid superporous hydrogel is used in the preparation of an artificial cornea. In this regard, specific embodiments embrace the incorporation of collagen and corneal fibroblasts into the pores of the superporous hydrogel.

Depending on the application, the superporous hydrogel of the invention can be used alone or in admixture of a pharmaceutically acceptable carrier in a pharmaceutical composition. Suitable formulations for use in the present invention are found in Remington: The Science and Practice of Pharmacy, Alfonso R. Gennaro, editor, 20th ed. Lippincott Williams & Wilkins: Philadelphia, Pa., 2000. Exemplary carriers include, e.g., water, saline, a buffer and the like. The compositions can also contain pharmaceutically acceptable auxiliary substances as required to approximate physiological conditions, such as pH adjusting and buffering agents, tonicity adjusting agents, wetting agents, detergents and the like.

The compositions of the invention can be formulated for any appropriate manner of administration, including for example, topical, subcutaneous implantation or intramuscular implantation depending on the site at which cells are to be delivered and the disease or condition be treated.

The present method of producing a hybrid superporous scaffold is simple, inexpensive, and versatile. Therefore, it can be applied to many tissue engineering applications including skeletal and soft tissue applications. For example, in addition to corneal regeneration, the hybrid superporous hydrogen can be used in bone tissue engineering. Indeed, many modifications can be made to the disclosed hydrogel to tailor it for a particular tissue. Hydrolytic linkages can be incorporated within the SPH to create degradable and non toxic by products over time. Drugs or molecules could be loaded within the SPH for controlled release situations. Both natural and synthetic materials can be altered to produce specifically desired properties. For example, a PEGDA-based polymer cannot withstand tensile forces including suturing. However, the PHEMA-PMMA copolymer of the present invention possesses sufficient tensile strength for suturing. Thus, one of skill would choose the superporous hydrogel of the present invention based on the desired properties.

The invention is described in greater detail by the following non-limiting examples.

### Example 1

### Materials & Methods

Cell Culture.

Two cell types, stem cells and committed cells, were analyzed. Human mesenchymal stem cells (MSCs) were maintained in Gibco's α-Minimal Essential Medium (with L-glutamine, without ribonucleosides, without deoxyribonucleosides) containing 15% fetal bovine serum (FBS), 1% L-glutamine, and 1% antibiotics. The HT-1080 human fibrosarcoma cell line was purchased from ATCC (Manassas, Va.). Fibroblasts were bathed in Dulbecco's Modified Eagle's Medium (DMEM) supplemented with 10% fetal bovine serum (FBS) and 1% antibiotics/antimycotics. Media was changed every two to three days to remove wastes and provide fresh nutrition. Cells were maintained at 37° C. in the presence of 5% CO₂ and 95% air. Cells were plated at a density of 3×10³ cells/cm² in tissue culture flasks until a 75-80% confluent monolayer was formed. Cells were passaged by incubating for 5 minutes with 0.25 mg/mL trypsin and replating at the above density. All cells used in the experiments herein were between passages 3 and 6. The method presented herein can be extended to other cell types. Cells can be either loaded into the scaffold prior to implantation or cells can be encouraged to migrate into the scaffold post-implantation.

Collagen Gel Preparation.

Rat tail collagen type I (BD Biosciences, San Diego, Calif.) was mixed with 0.1 N NaOH, 10× Hank's Balanced Salt Solution (HBSS), and 0.1 N acetic acid at a volume ratio of 3:2:1:1. This created a neutral pH collagen solution at a concentration of 1 mg/mL. If cell seeding was desired, cells were suspended in the collagen solution at a concentration of 1 millions cells/mL to encapsulate them in a 3-D network. Soaking a dehydrated SPH in this solution allowed uptake of cells and collagen within the pores. Collagen gelation was initiated by warming to 37° C. for 30 minutes. If pre-seeding with cells was not desired, the SPH was soaked in the collagen solution without cells. Again gelation occurred by warming to 37° C. for 30 minutes.

Superporous PEGDA Hydrogel Fabrication.

A 20% (w/v) PEGDA solution (500 μL) was combined with the following reagents: 60 μL of 10% PLURONIC PF-127, 30 μL of 20% TEMED, 20 μL of acrylic acid, and 23 μL of APS. The final volume was adjusted to 1 mL via addition of deionized water. The solution was heated for two minutes at 37° C. Subsequently, 200 mg of sodium bicarbonate was mixed in the solution, which created a foaming reaction resulting in a porous structure. The amount of sodium bicarbonate was varied from 100 to 300 mg to create differences in pore architecture. SPHs were rinsed in water to remove unreacted sodium bicarbonate crystals. To prevent pore collapse, the scaffolds were dehydrated in ethanol overnight. Scaffolds were then further dehydrated in a food dehydrator for 45 minutes. Cut sections were placed under UV light for 20 minutes to sterilize.

Superporous PHEMA-PMMA Hydrogel Copolymer Fabrication.

The gel solution used to create the porous PHEMA-PMMA copolymer included: 10% v/v methyl methacrylate (MMA) (Aldrich M55909), 45% v/v 2-hydroxyethyl methacrylate (HEMA) (Aldrich 525464), 5 mg pentaerythritol tetraacrylate (PETA), 2 mg ammonium persulfate (APS), 10 μL N,N,N′,N′-tetramethylethylenediamine (TEMED), 6% v/v dimethylformamide, and 22% deionized water. The gel was mixed until a viscosity comparable to soft chewing gum was achieved. Next, the gel was inserted into a mold. The product was polymerized at 37° C. in dry heat for 24 hours. Following polymerization, the gel was rinsed in deionized water for up to one week. For long term storage, water rinsed gels were desiccated at 37° C. in dry heat for 1 day.

Pore Formation.

The PHEMA sponge (U.S. Pat. No. 5,458,819) has been developed to create porous PHEMA scaffold. Porous PHEMA sponge results from phase separation as the dissolved monomers become polymers and fall out of solution. For the PHEMA-PMMA construct, dimethylformamide (DMF) and pentaerythritol tetraacrylate were added to the mixture of MMA and HEMA monomers. DMF was found to be an important process that facilitated dissolving PHEMA-PMMA polymers. Incorrect use of DMF led to destruction of pores. Use of pentaerythritol tetraacrylate as a crosslinker is unique in that it allowed PHEMA-PMMA crosslinking with desired mechanical properties. Stirring while polymerization takes place was another key step that led to formation of large pores. The gel-like solution develops a viscosity that is comparable to molasses and is flexible enough to be placed into a contact lens-shaped mold, mimicking the curvature of the cornea.

Creating the Hybrid Suturable Implant.

The process for forming an inter-penetrating collagen network in the porous PHEMA-PMMA can take up to 36 hours due to the slow expansion of PHEMA-PMMA in aqueous solution. First, the polymer construct was sterilized under ultraviolet light for 30 minutes. Collagen gel (1-5 mg/ml) was made using rat tail collagen type I (BD Bioscience; human collagen is just easily incorporated) according to the BD Bioscience protocol. Concentrations of 1-5 mg/ml or collagen appear to support cell seeding and attachment. The collagen gel solution was added to the cooled and dehydrated PHEMA-PMMA. The sample and an ice pack were covered to maintain cool temperature. The PHEMA-PMMA sample must remain cooled, approximately 2-8° C., throughout the collagen absorption process. After the PHEMA-PMMA fully expanded in the collagen solution, the construct, now referred to as the suturable hybrid implant, was incubated at 37° C. for 1 hour in a cell incubator to create the collagen gel.

Pore Architecture and Swelling Measurements.

A scanning electron microscope was used to picture the pore architecture of the SPH. SPHs made with varying amounts of sodium bicarbonate were imaged. Rapid swelling to large volumes is an important feature for this application. A swelling ratio, Q, was determined by comparing the mass of the swollen SPH to the mass of the dehydrated SPH. Dehydrated structures of varying pore sizes were soaked in water for at least 20 minutes. All SPHs were centrifuged at 1000 rpm for 3 minutes to remove air bubbles. SPHs were strained with a sieve, to remove excess water, and weighed. This mass represents the water accumulated in the pores as well as in the hydrogel structure itself. Subsequently, the SPHs were gently squeezed and blotted to remove water in the pores but maintain water in the hydrogel structure. By dividing the swollen weight by the initial weight, two swelling ratios, Q_{Total Water} & Q_{Hydrogel Water}, were obtained.

Q_{Total Water}=Weight_{Total Water}/Weight_{Dehyrated}

Q_{Hydrogel Water}=Weight_{Hydrogel Water}/Weight_{Dehydrated}

Cell Staining.

SPH constructs with and without collagen were employed as 3-D fibroblast scaffolds. Cells were loaded as described above and incubated for 24 and 48 hours. A focal adhesion kit (Chemicon, Temecula, Calif.) was used to visualize cell adhesion and retention. Rhodamine phalloidin stained microfilaments red and DAPI stained nuclei blue. A live/dead viability kit (Molecular Probes, Eugene, Oreg.) was used to show cells that were alive (green) versus dead (red). A Bio-Rad confocal microscope was used to image each of these structures.

Cell Migration.

In cases where pre-seeding with cells is not desirable, it was determined whether the hybrid scaffold was preferred for cell migration into the scaffold. A cellular SPH scaffolds with and without collagen were placed atop a monolayer of cells. Cell migration into the scaffold was monitored over 3 weeks. Cells were stained with live/dead viability and visualized with a confocal microscope.

Compressive Measurements.

Compressive modulus of the SPH scaffolds was determined by compressive testing. Water swollen SPHs were sandwiched between two pieces of glass lined with VELCRO (to prevent slippage) and compared to collagen swollen SPHs. Incremental weights were placed atop and the amount of strain that each SPH withstood was recorded. A stress versus strain curve was plotted to determine an estimate of compressive modulus.

Central Optic Hydrogel Synthesis.

A hydrogel solution was prepared by mixing 5% (w/v) of PEGDA in sterile PBS. A photoinitiator, IGRACURE 2959 (Ciba, Tarrytown, N.Y.) was added to the PEGDA solution for a final concentration of 0.025% w/v. Cell viability was assessed in response to photoinitiator, UV light exposure, and PEGDA concentration. IGRACURE 2959 was the least toxic photoinitiator to mammalian cells. A concentration of 0.03% (w/v) or less is considered optimal (Williams, et al. (2005) Biomaterials 26(11):1211-8). Placing this solution under UV light (365 nm, 4 mW) for 10 minutes created a clear polymerized gel. The gel was soaked in fresh PBS to remove unreacted monomers and initiator.

### Example 2

### Swelling Ratio

Since collagen begins to gel quickly after pH neutralization, immediate upload into the SPH was necessary to facilitate uniform distribution throughout the SPH. Since the SPH fabrication method created interconnected macrosized pores, swelling occurred in less than 1 minute. Soaking the SPH in a collagen solution allowed natural materials to enter the pores easily and rapidly via capillary action. Thus, wherein preseeding with cells is desired, cells can be suspended in the collagen solution just prior to uptake. Swelling was determined by the degree and size of interconnected pores. SEM analysis of pore structure in three SPHs created with 100, 200, and 300 mg of sodium bicarbonate revealed two types of pores: larger pores which appeared similar in size and shape in each of the SPHs and smaller pores, which formed the interconnection pathways. It was apparent that increasing the amount of sodium bicarbonate resulted in an increased number of interconnection pores. This indicated that the differences in pore architecture caused differences in swelling ratios. Swelling ratios, Q, were determined for SPHs of varying pore sizes (i.e., using 100, 200 or 300 mg sodium bicarbonate). This analysis indicated that QTotal Water increased as more sodium bicarbonate was used. However, QHydrogel Water had no appreciable difference with different pore sizes. This indicated that differing amounts of sodium bicarbonate altered pore structure, but the amount of hydrogel in each SPH remained the same. This phenomenon is important to note for applications in which it may be desirable to load molecules within the hydrogel structure itself. In addition, QTotal Water hovered around 100, indicating that the SPH was capable of incorporating about 100 times its dried weight. Therefore, any long term increases in weight due to cell proliferation or ECM production should not be barriers to long term stability.

Rapid uptake into the SPH did not incur cell injury. Indeed, a live/dead viability stain performed one day after cell loading showed that MSC cells were alive and spreading. CALCEIN AM crossed the cell membrane of live cells and fluoresced green while ethidium homodimer only entered dead cells and fluoresced red. Minimal dead cells were detected. This promised to be an effective and efficient method of cell loading that did not require the use of external forces or compromise cell viability.

While it has been suggested that a small pore size within a narrow range is essential to 3-D cell behavior within scaffolds, the results herein indicate SPH hybrid technology eliminates the need for precise control of pore size or shape with respect to 3-D cell adhesion. The instant method is unique in that noncovalent binding and a lack of intimate contact between scaffold materials separates the cellular microenvironment from the supporting SPH. When cells are in contact with PEGDA, despite the presence of collagen, cells are not able to spread out. Therefore spatial and temporal separation of the two materials is necessary for optimal cell behavior.

As evident in SEM images of the pore structure, shape and size are non uniform. However, observation of cell morphology and adhesion in the SPH-collagen gel showed similarity to purely 3-D collagen images. For that reason SPH pore structure is not a factor in cell behavior because cells do not contact the SPH. Cells in the hybrid matrix appear to be only embedded in the collagenous portion. So long as the pores are interconnected to assure uniform distribution and effective nutrient and waste diffusion, the instant hybrid superporous hydrogel does not require the stringent requirements of other systems to create a natural 3-D cell microenvironment. Thus, the hybrid hydrogel of this invention is more convenient and better mimics natural living systems that generally lack the uniformity imposed by engineered constructs.

### Example 3

### Adhesion Staining

In preseeded scaffolds, it was observed that collagen encouraged fibroblast spreading in 3-D and formation of stress fibers. Scaffolds without collagen housed clumped, round cells that were incapable of attaching to the scaffold. PEGDA is intrinsically resistant to adhesion. Thus, a lack of ECM cell binding sites in non-collagenous scaffolds was presumed to be responsible for the round morphology. After 48 hours, scaffolds without collagen were completely acellular. Having nothing to attach to, cells tended to migrate out of the scaffold and attach to the tissue culture plate below.

In contrast, collagen loaded scaffolds showed cell retention within the scaffold and few if any cells attached to the plate below. Collagen within the hydrogel pores greatly enhanced cell spreading and retention in a 3-D manner. The microfilament stress fibers were clearly observed, indicating that cell adhesion was mediated by integrin binding sites available in collagen, leading to formation of focal adhesion. Corneal fibroblasts have been shown to express a variety of β1 family integrins, which can bind to collagen. In scaffolds without collagen, β1-integrin was distributed uniformly around the periphery of the cell. However, in collagen-filled scaffolds, the β1-integrins were punctuated and clustered at sites of focal adhesions. Addition of antibodies against β1-integrins prevented cells from attaching and spreading on the collagens.

In addition to enhancing cell spreading and retention when embedded in the collagen of the pores, it was demonstrated that this hybrid scaffold also enhances surrounding cell migration into the scaffold by virtue of an open pore structure and collagen binding. Acellular SPH scaffolds with and without collagen were placed on top of a monolayer of fibroblasts. Over 3 weeks, tremendous cellular ingrowth was observed into scaffold with collagen. The scaffold without collagen remained acellular. This demonstrated that pores alone are not sufficient for cellular ingrowth, and the incorporation of collagen greatly enhances this scaffold as an ideal tissue engineering scaffold. Good cell ingrowth is necessary for in vivo implantation so that host cells can migrate into the scaffold and form a strong integration with the surrounding tissue. This is also a conduit for nerve and blood vessel ingrowth which may be necessary for long term survival of the implant.

### Example 4

### Mechanical Measurements

Compressive tests to determine compressive modulus indicated that the SPH was significantly more compressive than nonporous PEGDA alone. The average compressive modulus for the SPH without collagen was 1 kPa. When compared to nonporous PEGDA, it was observed that the SPH was able to withstand much higher compressive forces. This may be of importance in situations where the implant is subjected to high compressive forces. The addition of collagen in the SPH did not have a significant impact on the bulk modulus. Therefore, the instant hybrid hydrogel can maintain a high compressive modulus overall, without subjecting embedded cells to these conditions, since they are only exposed to the much softer collagen microenvironment.

Mechanical measurements were also performed to test the strength of the porous PHEMA-PMMA hybrid scaffold. Hydrated porous PHEMA-PMMA constructs were tested for tension using a custom designed testing device (Test Resources, Shakopee, Minn.). Samples were tested using a load cell that is fatigue-rated for 75 g of force (0.735 N) in tension. The applied forces were calibrated using calibration weights (Rice Lake Weighing System, Fisher Scientific). The force measurements were observed and reported by the vendor's 100LM software. Less than 1% error was observed in the 0.3 to 0.735 N range. Each sample was tested for 30 cycles at strain rate of 0.1 Hz. A strain of 5% was used to pre-condition samples. The elastic modulus was calculated using 20% strain. Samples were measured to rupture using up to 150% strain. Stress was computed by dividing the load output of the testing machine by the cross sectional area of the sample to which force was applied (Stress=Force/Area). The elastic modulus was estimated by determining the slope of the linear portion of the stress vs. strain curve. Standard deviation was used to determine error for the elastic modulus.

### Example 5

### Optical Properties

The central optic of an artificial cornea should be clear and have an appropriate refractive index. To demonstrate the use of PEGDA in the central core of an artificial cornea, qualitative and quantitative analyses of a 5% PEGda was carried out. For qualitative analysis, written text was viewed with and without an overlying hydrogel. Optical properties such as light transmission and refractive index of the central core were determined using a UV-Vis spectrophotometer and refractometer, respectively. The percent of light transmittance was measured in reference to PBS at wavelengths ranging from 200 nm to 1000 nm. All measurements were made in triplicate. The refractive index of the central optic is measured using a refractometer.

The results of these analyses indicated that a 5% hydrogel yielded excellent optical properties for use as a central optic. For example, clarity of written text viewed with or without an overlying hydrogel was observed to be similar. A quantitative study using UV-Vis spectrophotometry revealed high light transmittance over a broad range of wavelengths. For example, the average transparency at 550 nm was 90%. In addition, the refractive index was approximately 1.34 (˜5 brix) which is only slightly less than the natural cornea, 1.37.

### Example 6

### Artificial Cornea Fabrication

Based on the natural corneal architecture, the anterior surface of a hybrid hydrogel matrix is coated with epithelial cells to encourage host epithelialization to regenerate the protective and nutrient absorbing qualities of the epithelium. Below the epithelium, similar to Bowman's layer, a thin layer of nonporous PEGDA is used to separate the epithelium from the underlying stroma. PEGDA discourages cell binding and keeps cell types localized. Within the stromal skirt, collagen and cells are surrounded by PEGDA, a hydrogel that is capable of retaining large amounts of water to maintain an appropriate shape and hydration level. The hybrid superporous skirt is designed to allow maximal host cell integration through the pores and attachment to cell adhesion sites. The central core is kept free of collagen to maintain optical transparency.

The production of a biodegradable, porous, cell-based, tissue-engineered cornea has now been accomplished. As a first step in producing such an artificial cornea, an SPH disc was soaked in a fibroblast-collagen solution. As a control, SPHs were also soaked in cell solutions without collagen. Submersion in liquid caused rapid swelling of the SPH and uptake of collagen and cells within the pore network. Collagen fibers were then thermogelled. The result was a collagenous microenvironment dispersed throughout a mechanically stable hydrogel. A central hole was carved out with a 5 mm trephine. The central hole was filled with a nonporous, optically clear PEGDA macromer solution. The nonporous PEGDA solution diffused into the immediate periphery and spread along the bottom surface of the SPH and deposited a thin layer of nonporous PEGDA on the anterior surface. The nonporous PEGDA was photopolymerized into this irregular shape. The anterior surface could then be modified with collagen so that epithelial cells could be attached for proliferation on the top surface.

To epithelialize the surface of the hybrid hydrogel, a water soluble heterobifunctional cross-linker such as sulfo-SANPAH (PierceNet) can be used to attach collagen type 1 to the surface of PEGDA. The N-hydroxysuccinimide group attaches to collagen proteins while the phenyl azide group photoreactively inserts into PEGDA. The presence of covalently bound collagen can be imaged with second harmonic generation using a multiphoton microscope. Compared to the stroma, the collagen on the surface will be a thin layer ( 1/10 of the concentration). This thin layer of collagen is expected to support the growth of corneal epithelial cells as determined by, e.g., intermediate filament keratin-3 and keratin-12 expression. It is contemplated that this layer can be optimized for sufficient cell attachment while maintaining clarity. Clarity of the collagen layer can be assessed via UV-Vis spectrophotometry.

Corneal epithelial cultures can obtained from rabbit limbal tissue of approximately 3×2 mm at the time of surgery. The tissue is treated with dispase (10 mg/ml) at 4° C. overnight to disrupt the basement membrane. The epithelial sheets are peeled off and digested in 0.25% trypsin-EDTA at 37° C. for 5-10 minutes. Cells are washed and resuspended in keratinocyte serum-free medium (KSFM, Invitrogen), and plated on collagen-coated tissue culture plates. When cells reach 80% confluency, epithelial cells are trypsinized and plated on the hybrid scaffold.

Five major forces have been shown to act on a contact lens (i.e., atmospheric pressure, hydrostatic pressure of the postlens tear film, surface tension of the prelens tear film, lens weight and lid force (Leonardi, et al. (2004) Invest. Ophthalmol. Vis. Sci. 45(9):3113-7). Thus, it can be assumed that an artificial cornea will be exposed to similar forces post-implantation. Based on this rationale, assessment of the mechanical properties of the superporous hybrid scaffold can be determined. For example, the Young's modulus can be measured using an atomic force microscope (AFM) and the Hertz model.

The Young's modulus is calculated using Hertz′ model:

$${F}_{sphere} = \frac{4}{3} \cdot \frac{E}{( 1 - {v}^{2} )} \cdot {δ}^{3 / 2} \cdot \sqrt{R} = k \cdot d$$

where F is indenting force; R is attached bead radius; δ is indentation, assuming that δ<<R; E is Young's modulus, ν is Poisson ratio (0.5 for incompressible sample); k is cantilever's spring constant, and d is cantilever's deflection. This model assumes a homogeneous, isotropic, semi-infinite elastic material. Also the surface should be flat, a conical or spherical tip should be used, and the indenter material should be much stiffer than the sample.

The Young's modulus of a human donor cornea was reported as 1.3 MPa (Wollensak, et al. (2003) J. Cataract. Refract. Surg. 29(9):1780-5). The Young's modulus of the individual components of the artificial cornea were measured and found to be 2 MPa for a 5% PEGDA gel and ˜1 kPa for a 1 mg/mL collagen gel. However, the contribution of collagens to the overall mechanical properties is expected to be minimal.

In addition, the shear modulus of a hybrid scaffold can be determined using elastography. Elastography is a magnetic resonance-based technique that measures mechanical properties by propagating an electromagnetic wave through the material (Zerbe, et al. (2006) supra). The system is ideal for mechanical measurements of soft tissues and complements the AFM measurements. This non-destructive 3D imaging technique can also measure the diffusion of water, which is a direct indication of the tissue structure and viability. When cells swell or cell membranes rupture, for example, water diffusion is more rapid due to fewer physical barriers.

### Example 7

### Cell Migration into the Porous PHEMA-PMMA Hybrid Scaffold

Cell migration and cell viability of the porous PHEMA-PMMA hybrid scaffold were tested using a commercially available live/dead cell assay. For cell migration studies, acellular porous PHEMA-PMMA scaffolds with and without collagen were placed on the top of a monolayer of fibroblasts. After a pre-determined period of incubation time, the scaffolds were removed and the cells in the scaffold were assayed. In control experiment in which interpenetrating collagen network was not engineered into the scaffold, no cells were found. This indicates the lack of cell migration into the scaffold. In contrast, when interpenetrating collagen network was engineered into the scaffold, strong evidence for cell migration was readily obtained. Not only have cells migrated into the scaffold, but the cells were also stained live-clearly demonstrating the bioactivity of the porous PHEMA-PMAA hybrid scaffold by inducing cells to move up into the scaffold and attach to the collagen network for survival. It should be anticipated that the cells migrated into the scaffold secrete proteins and other molecules necessary to construct their own extracellular matrix, which serves as a definitive marker for tissue integration.

### Example 8

### Biocompatibility of an Artificial Cornea in a Rabbit Model

In vivo biocompatibility in a rabbit model is used to evaluate the degree of host-prosthesis integration, epithelialization, stability, and clarity of the artificial cornea. It is expected that pre-seeding the porous skirt with collagen and fibroblasts will significantly enhance the rate and degree of integration and lead to long-term stability of the implant.

The first step prior to implanting the artificial cornea is to obtain autologous corneal fibroblasts from the rabbit. This is done through a small corneal biopsy from the peripheral cornea. The corneal biopsy tissue is then digested in 1 mg/ml of collagenase overnight at 37° C. The cells are washed and then plated in DMEM supplemented with 10% FCS (Sigma-Aldrich, St. Louis, Mo.), 4 mM L-glutamine and 1% antibiotic solution (Invitrogen-Gibco). After a period of 10-14 days in culture, the fibroblasts are trypsinized and dispersed in collagen solution and incorporated into the superporous skirt of the artificial corneas.

The artificial corneas either seeded with fibroblasts or un-seeded (control) are surgically implanted into the rabbit cornea in a two stage procedure. In the first stage the artificial cornea is implanted as a partial thickness replacement keeping the anterior cornea of the rabbit as a protective flap. Specifically, using a Moria microkeratome (designed to make LASIK flaps) a 130 μm flap of approximately 10 mm diameter is created. This in effect slices the cornea horizontally. The posterior aspect of the cornea which is now exposed is trephinated using an 8 mm trephine and the corneal implant composed of a 4 mm core and 2 mm skirt is sutured in place using interrupted 10-0 nylon sutures. The control rabbits receive the same implants but without any cells imbedded in the skirt. The anterior flap is placed back on top of the implant and sutured to the peripheral cornea using interrupted dissolvable 10-0 vicryl sutures.

In stage two, the portion of the anterior flap which covers the clear zone of the implant is removed and the artificial cornea functions as a full-thickness replacement. The rationale for this staged procedure is to maintain the integrity of the cornea while allowing time for integration to take place. Specifically, animals are returned to the operating room 2 months after the initial implantation. The rabbits are placed under general anesthesia and the central 4 mm of the anterior flap covering the clear zone of the implant is trephinated and removed.

Follow-ups will be performed daily on each rabbit for the first week after surgery and then 2-3 times a week for evidence of complications such as melting, aqueous leakage, extrusion, infection, retroprosthetic membrane formation, retinal detachment, or proliferative vitreoretinopathy. Examinations will include slit lamp biomicroscopy to ensure that corneas are optically clear, and sodium fluorescein staining to assess integrity and barrier function. Intraocular pressure measurements are also taken to determine if the implants are interfering with aqueous humor flow. Indirect ophthalmoscopy is used to examine the posterior segment. All animals are followed for 6 weeks to determine the short term outcome and then up to 6 month for long-term studies.

The bio-integration and bio-compatibility of the artificial corneas will be evaluated histologically at 1 week, 2 weeks, 6 weeks, 3 months, and 6 months. Three pairs of rabbits (one experimental and one control) will be used for histopathology at each time point. The eyes will be subjected to routine histology and immuno-staining to evaluate the degree of epithelialization over the core, fibroblast ingrowth into the skirts, and capsule formation around the device. Immunostaining against smooth muscle actin will be used to identify fibroblasts in the superporous skirt. The number and extent of fibroblast penetration into the implant will be graded by masked observers using serial sections starting from the periphery towards the center. The expression of collagen type I, β1-integrin and focal adhesion complexes will likewise be evaluated. The results will be compared between the two groups.

The integration of the artificial corneas will be mechanically tested according to conventional methods (Lee, et al. (2000) Arch. Ophthalmol. 118(12):1673-8). These measurements will be performed on intact eyes that are enucleated after euthanasia. The intraocular pressure will be progressively increased inside and the pressure at which the host-prosthesis begins to leak will be recorded. These measurements will be done three pairs of eyes (one control, one experimental) for each time point starting at 6 weeks, then 3 months, and 6 months.

As with any prosthetic device, there is a possibility of non-integration with secondary tissue necrosis or extrusion. It is contemplated that constructs pre-seeded with cells will significantly enhance integration. Alternative strategies to promote integration include embedding the skirt with sustained release growth factors such as TGF-beta to promote fibrovascular ingrowth. Another potential problem is membrane formation around the device especially behind the core (Hicks & Hamilton (2005) Cornea 24(6):692-8). In clinical settings, these membranes can typically be removed by YAG laser, however additional strategies for surface modification may also be used to inhibit membrane formation.

What is claimed is:

1. A scaffold for corneal regeneration or replacement comprising a superporous hydrogel matrix comprising a poly(2-hydroxyethyl methacrylate) (PHEMA) and poly(methyl methacrylate) (PMMA) copolymer, and a cell adhesion agent in the pores of said superporous hydrogel matrix.

2. The scaffold of claim 1, wherein the cell adhesion agent is collagen.

3. The scaffold of claim 1, wherein the pores of the superporous hydrogel matrix have an average diameter of greater than 100 μm.

4. The scaffold of claim 1, wherein the pores of the superporous hydrogel matrix are connected to one another to form an open channel system.

5. The scaffold of claim 1, wherein the equilibrium swelling capacity of the superporous hydrogel matrix ranges from 50 to 500 g/g.

6. The scaffold of claim 1, wherein the superporous hydrogel matrix has an ultimate tensile strength of about 80 kPa or more.

7. The scaffold of claim 1, wherein the copolymer is formed by dissolving 2-hydroxyethyl methacrylate (HEMA) and methylmethyacrylate (MMA) monomers in a solvent and polymerizing the monomers at a temperature below 45° C. in the presence of a pore-forming agent.

8. A suturable implant comprising the scaffold of claim 1, wherein said implant forms the skirt of a core-skirt keratoprosthesis for implantation into the cornea.

9. A method for producing the scaffold of claim 1 comprising
(a) dissolving 2-hydroxyethyl methacrylate (HEMA) and methylmethyacrylate (MMA) monomers in a solvent;
(b) polymerizing the monomers at a temperature below 45° C. in the presence of a pore-forming agent to form a superporous hydrogel matrix; and
(c) integrating a cell adhesion agent into the pores of the superporous hydrogel matrix.

10. The method of claim 9, wherein the cell adhesion agent is collagen.
